(19) United States
(12) Patent Application Publication
(10) Pub. No.: US 2009/0041688 A1
(43) Pub. Date: Feb. 12, 2009

(54) Sunless tanning compositions comprising certain substituted polyamine compounds and methods of use

(76) Inventors: Olga Dueva-Koganov, White Plains, NY (US); Xian-Zhi Zhou, Leonia, NJ (US); Shaun Barker, Brooklyn, NY (US); John Jennings, Moycullen (IE)

Correspondence Address: JoAnn Villamizar;Ciba Corporation/Patent Department, 540 White Plains Road, P.O. Box 2005, Tarrytown, NY, 10591, US

(21) Appl. No.: 12/220,158

(22) Filed: Jul. 22, 2008

Related U.S. Application Data
(60) Provisional application No. 60/962,359, filed on Jul. 27, 2007.

(51) Int. Cl. A61K 8/72; A61Q 19/04

(52) U.S. Cl. 424/59

(57) ABSTRACT

The present invention is directed to cosmetic and/or dermatological compositions for enhancing the rate of tanning human skin with sunless tanning compositions and providing the added benefit of simultaneously providing protection from ultraviolet light radiation. More particularly, the present invention is directed to a sunless tanning composition comprising a sunless tanning agent, a substituted polyamine compound and a cosmetically acceptable adjuvant. Methods of use of the instant compositions are disclosed as well.

This application claims benefit of U.S. provisional app. No. 60/962,359, filed Jul. 27, 2007, the contents of which are incorporated by reference.

### FIELD OF INVENTION

The present invention is directed to cosmetic and/or dermatological compositions for enhancing the rate of tanning human skin with sunless tanning compositions and providing the added benefit of simultaneously providing protection from ultraviolet light radiation. More particularly, the present invention is directed to a sunless tanning composition comprising a sunless tanning agent, a substituted polyamine compound and a cosmetically acceptable adjuvant. Methods of use of the instant compositions are disclosed as well.

### BACKGROUND OF THE INVENTION

A sun-tanned appearance is a symbol of a healthy, dynamic, and active life. Yet, the damaging effects of sunlight and other sources of ultraviolet radiation on the skin are well documented. These effects are cumulative and potentially serious, and include sunburn, skin cancer, and premature aging of the skin. These effects associated with exposure to ultraviolet radiation are more fully discussed in DeSimone, “Sunscreen and Suntan Products”, Handbook of Nonprescription Drugs, 7th Ed., Chapter 26, pp. 499-511 (American Pharmaceutical Association, Washington, D.C.; 1982); Grove and Forbes, “A Method for Evaluating the Photoprotection Action of Sunscreen Agents Against UV-A Radiation”, International Journal of Cosmetic Science, 4, pp. 15-24 (1982); and U.S. Pat. No. 4,387,089, DePolo, issued Jun. 7, 1983; the disclosures of all of which are incorporated herein by reference in their entirety.

Sunscreens are the most common agents used for sun protection. However, sunscreens also have the disadvantage of preventing or greatly diminishing the cosmetically desirable tanning response. Thus, if an individual uses a sunscreen for protection from ultraviolet radiation, he or she does so at the expense of foregoing a tanned appearance. Furthermore, even if an individual is willing to accept the risks associated with exposure to ultraviolet radiation to obtain a tan, there are situations in which it may not be practical or even possible to do so because of time constraints, weather conditions, etc. Therefore, it would be highly desirable to develop products for providing a tanned appearance to the skin, whenever desired without the need for exposure to ultraviolet radiation. Furthermore, it would be desirable and advantageous to have a sunless tanning product that would provide a tanned appearance to the skin and provide protection from ultraviolet radiation in the form of a sunscreen and/or UV absorber.

Most of the cosmetic products intended for artificially tanning the skin are based on carbonyl derivatives allowing, by interaction with the amino acids of the skin, the formation of colored products, among which mention is made of mono- or polycarbonyl compounds, for example, isatin, alloxan, ninhydrin, glyceraldehyde, mesotartaric aldehyde, glutaraldehyde, erythrulose and dihydroxyacetone (DHA).

These compounds react with free amino groups in the skin in a Maillard reaction to give brown-colored substances in the stratum corneum. This reaction is complete after 4 to 12 hours. The tanned appearance achieved cannot be washed off and is removed only with normal skin desquamation, i.e. it takes approximately 5 to 15 days until the skin is completely decolored.

It is generally known that dihydroxyacetone, when applied topically to human skin, will produce a tanned appearance, i.e. an artificial tan. U.S. Pat. No. 4,708,865, to Turner, issued Nov. 24, 1987 describes the use of hydro-alcoholic solutions of dihydroxyacetone for tanning the skin; U.S. Pat. Nos. 4,466,805, to Welters, issued Aug. 21, 1984 describes hair and skin coloring formulations containing dihydroxyacetone; and 2,949,403, to Andreadis et al., issued Aug. 16, 1960 describes artificial tanning formulations containing dihydroxyacetone in an oleaginous base.

Dihydroxyacetone is relatively sensitive to heat, light, and moisture. It is known that products containing dihydroxyacetone generally have a short shelf life, tending to darken and develop disagreeable off-odors over time, with a concomitant loss of product performance.

Dihydroxyacetone can react with other ingredients in a formulation, especially with nitrogen-containing compounds, such as amines, amino acids, and the like. In fact, without being limited by theory, dihydroxyacetone is believed to provide an artificial tan to human skin by its reaction with the nitrogen containing proteins of the skin. See L. Goldman et al., “Investigative Studies with the Skin Coloring Agents Dihydroxyacetone and Glyoxal”, The Journal of Investigative Dermatology, vol. 35, pp. 161-164 (1960); E. Wittgenstein et al., “Reaction of Dihydroxyacetone (DHA) with Human Skin Callus and Amino Compounds”, The Journal of Investigative Dermatology, vol. 36, pp. 283-286 (1961); and A. Meybeck, “A Spectroscopic Study of the Reaction Products of Dihydroxyacetone With Amino Acids”, J. Soc. Cosmet. Chem., 25-35 (1977); all of which are incorporated by reference herein in their entirety. These stability and incompatibility problems have limited the scope of artificial tanning products in the past.

Many artificial tanning products also have the disadvantage of not providing the desired control over color development of the tan. Artificial tans are often either too light or too dark, and tend to be too orange, uneven, or unnatural in appearance. Artificial tans often take too long to develop—sometimes as long as several hours or overnight. Also, it is known that some individuals are “non-reactors” or “inadequate reactors” in that these individuals do not develop an artificial tan with dihydroxyacetone or develop an artificial tan to only a slight extent. Therefore, a need exists to develop artificial tanning compositions that are chemically and physically stable, are aesthetically pleasing to use, that provide improved color development characteristics, and that provide an artificial tan for non-reacting and inadequately reacting individuals.

It is well known that various chemical compounds can be used to modify or enhance the tanning reaction obtained with dihydroxyacetone on human skin. Examples of such compounds include amino acids. See, e.g. Kawashima et al., “Nonenzymatic Browning Reactions of Dihydroxyacetone With Amino Acids or Their Esters”, Agric. Biol. Chem. 44(7), 1595-1599 (1980), and M. F. Bobin et al., “Effects of Color Adjuvants On the Tanning Effect of Dihydroxyacetone”, J. Soc. Cosmet. Chem., 35 265-272 (August 1984), both of which are incorporated by reference herein in their entirety.

It is generally known that the reaction of dihydroxyacetone with amino acids is difficult to control and has been an obstacle to successfully using amino acids in combination with dihydroxyacetone in an artificial tanning composition. For example, when dihydroxyacetone is formulated with an amino acid, the composition tends to undergo an unacceptable discoloration reaction during storage. A possible solution to this problem is to formulate the dihydroxyacetone separately from the amino acids and to deliver the separate formulations either sequentially from separate containers or simultaneously from a dual-chambered dispensing device. However, these alternatives are inconvenient, cumbersome, and expensive to implement and use. See, e.g. European Patent No. 527,864, assigned to Unilever, published Jun. 23, 1993.

U.S. Pat. No. 5,603,923 discloses artificial tanning compositions comprising dihydroxyacetone, certain amino acids and stabilizing salts, the disclosure of which is herein incorporated by reference in its entirety.

US 2005/089486 discloses self tanning compositions comprising a self tanning compound and an amine potentiator loaded on polymeric microparticles, the disclosure of which is herein incorporated by reference in its entirety.

WO 02/055052 discloses cosmetic compositions comprising a self tanning agent and at least one amphiphilic polymer containing a sulfonic group.

U.S. Pat. No. 5,503,824 discloses skin tanning compositions comprising dihydroxyacetone and aminosubstituted silicone compounds, the disclosure of which is herein incorporated by reference in its entirety.

U.S. Pat. No. 5,232,688 discloses self tanning cosmetic compositions comprising dihydroxyacetone and polyacrylamide, the disclosure of which is herein incorporated by reference in its entirety.

FR 2,779,958 discloses self tanning compositions comprising dihydroxyacetone and at least one N-substituted benzazole derivative.

FR 2,746,312 discloses self tanning compositions comprising dihydroxyacetone and benzotriazole derivatives containing siloxane groups.

U.S. Pat. No. 5,705,145 discloses skin tanning compositions comprising dihydroxyacetone and certain azole structures, the disclosure of which is herein incorporated by reference in its entirety.

FR 2,698,267 discloses self tanning compositions containing dihydroxyacetone and crosslinked copolymers of acrylamide-2-acrylamido-3-methylpropane sulfonic acid.

EP 1,277,461 discloses self tanning compositions containing triazine derivatives and dihydroxyacetone.

EP 1,277,460 discloses self tanning compositions containing benzoazoyl, benzodiazoyl, or benzotriazolyl derivatives and dihydroxyacetone.

DE 198 08 066 discloses a sunless tanning composition comprising dihydroxyacetone and at least dibenzoylmethane derivative and at least one methylidene camphor derivative.

JP 2001213747 discloses cosmetic compositions comprising dihydroxyacetone and dicarboxylic acid diesters for self tanning.

JP 06227937 discloses sunless tanning compositions containing dihydroxyacetone and lysine.

US 2007/0003496 discloses sunless tanning compositions containing dihydroxyacetone and amphoglycinate derivatives, the disclosure of which is herein incorporated by reference in its entirety.

U.S. Pat. No. 6,616,918 discloses self tanning compositions containing a self tanning agent and an N-acylamino acid ester, the disclosure of which is herein incorporated by reference in its entirety.

US 2004/0228810 discloses sprayable sunless tanning solutions with sunscreen protection, the disclosure of which is herein incorporated by reference in its entirety.

WO 97/33560 discloses sunless tanning compositions comprising dihydroxyacetone and hydroxysilyl substituted ethylene diamine derivatives.

US 2004/0013617 discloses sunless tanning compositions comprising dihydroxyacetone and at least one phospholipid and at least one nonionic surfactant and at least one amphoteric surfactant, the disclosure of which is herein incorporated by reference in its entirety.

US 2003/0108495 discloses cosmetic compositions containing precursors to artificial tanning agents, the disclosure of which is herein incorporated by reference in its entirety.

US 2003/0068286 discloses self tanning compositions comprising dihydroxyacetone and a benzaldehyde derivative, the disclosure of which is herein incorporated by reference in its entirety.

U.S. Pat. No. 6,706,257 discloses sunless tanning products comprising dihydroxyacetone and methylsulfonylmethane, the disclosure of which is herein incorporated by reference in its entirety.

U.S. Pat. No. 6,699,462, discloses sunless tanning compositions comprising sorghum extracts, the disclosure of which is herein incorporated by reference in its entirety.

U.S. Pat. No. 6,344,185 discloses self tanning compositions comprising dihydroxyacetone and polyester-type polymers, the disclosure of which is herein incorporated by reference in its entirety.

U.S. Pat. No. 6,171,605 discloses self tanning compositions containing dihydroxyacetone and propolis extracts, the disclosure of which is herein incorporated by reference in its entirety.

A. Muller discloses in Cosmetics and Toiletries, 1992, 107, 125-132 sunless tanning compositions comprising dihydroxyacetone and tyrosine derivatives.

Tessier et al. in Biochem. J. (2003) 369, 705-719 disclose triosidines as novel Maillard reaction products between some amino acid residues and dihydroxyacetone.

There is thus increasing demand for fast-acting self-tanning products that give a coloration close to that of natural tanning and provide protection from ultraviolet radiation.

Surprisingly and advantageously, the inventors have found that the use of certain substituted polyamine compounds improves the stability and coloration of compositions comprising a self-tanning agent. The colorations obtained are more chromatic and more stable over time, and also show good water resistance and good homogeneity.

### SUMMARY OF THE INVENTION

The present invention is directed to a cosmetic and/or dermatological composition comprising: i) a sunless tanning agent, ii) a substituted polyamine compound, and iii) a cosmetically acceptable adjuvant. Methods of use of the instant compositions are disclosed as well.

### DETAILED DISCLOSURE

The present invention is directed to a cosmetic and/or dermatological composition comprising:
i) a sunless tanning agent,
ii) a substituted polyamine compound of formula (I)

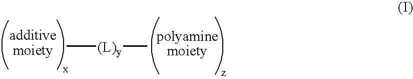

wherein
x and y are each independently greater than or equal to 1;
z is 1 to 5;
L is independently of each other a direct bond or a chemical linking group;
additive moiety is independently selected from the group consisting of antioxidant, ultraviolet light absorber, hindered amine light stabilizer, hydroxylamine stabilizer, nitrone stabilizer, amine oxide stabilizer, and benzofuranone stabilizer and or mixtures thereof;
polyamine moiety is independently selected from the group consisting of polyethyleneimine, polyaminoacids, polyvinylamine, and oligomeric ethylene amines or mixtures thereof; and
iii) a cosmetically acceptable adjuvant,
   with the proviso that in formula (I) of component ii) the additive moiety is covalently attached to said polyamine moiety through said chemical linking group.

For the purposes of the present invention, the expression “self-tanning agent” or “sunless tanning agent” means an agent which, when applied to the skin, gives a tanning effect that is more or less similar in appearance to that which may result from a prolonged exposure to sunlight (natural tan) or a UV lamp.

The compositions according to the present invention also have the advantage of having improved cosmetic properties: they give the skin a feeling of softness and freshness, and prevent the skin from drying out and also from having an excessively greasy feel.

An embodiment of the present invention is also the use of the composition according to the invention as a composition for tanning or browning the skin; and a cosmetic process for tanning or browning the skin such that it consists in applying to the skin an effective amount of a composition according to the invention.

Another embodiment of the instant invention is a method for tanning or browning the skin such that it consists in applying to the skin an effective amount of a composition according to the invention.

The invention also relates to the use of these compositions for giving the skin a coloration close to that of natural tanning of the skin.

The invention also relates to the use of these compositions for giving the skin a coloration close to that of natural tanning of the skin.

Finally, the present invention also relates to the use of at least one substituted polyamine of formula (I) in compositions for artificially tanning and/or browning the skin, containing at least one self-tanning agent, in order to improve the coloration and/or stability of the self-tanning agent.

The compositions in accordance with the invention give an artificial coloration that is close to that of natural tanning in a short space of time. Thus, an immediate coloration is obtained, which allows visualization of the application and, consequently, better uniformity in the spreading of the composition onto the skin and thus of the resulting coloration. Furthermore, the artificial coloration obtained on the skin according to the invention is extremely close to that of a natural tan.

Other characteristics, aspects and advantages of the invention will become apparent on reading the detailed description that follows.

The sunless tanning agents of component i) are generally chosen from mono- or polycarbonyl compounds such as, for example, isatin, alloxan, ninhydrin, glyceraldehyde, mesotartaric aldehyde, glutaraldehyde, erythrulose, pyrazoline-4,5-dione derivatives as described in patent applications FR 2 466 492 and WO 97/35842, dihydroxyacetone (DHA), and 4,4-dihydroxypyrazoline-5-one derivatives as described in patent application EP 903 342.

In another embodiment of the instant invention, the sunless tanning agent of component i) is selected form the group consisting of isatin, alloxan, ninhydrin, glyceraldehyde, mesotartaric aldehyde, glutaraldehyde, erythrulose, a pyrazoline-4,5-dione derivative, dihydroxyacetone (DHA) and a 4,4-dihydroxypyrazoline-5-dione derivative or mixtures thereof.

In another embodiment of the instant invention, the sunless tanning agent of component i) is dihydroxyacetone (DHA).

The additive moiety is for example independently selected from the group consisting of antioxidant, ultraviolet light absorber, hindered amine light stabilizer, hydroxylamine stabilizer, nitrone stabilizer, amine oxide stabilizer, and benzofuranone stabilizer moieties.

The terms x, y independently may be for example 1 to about 200, 1 to about 100, 1 to about 50, for instance 1 to about 25, 1 to about 10 or 1 to about 5.

The term z is for example 1, 2, 3 or 4. Particularly, z is 1 or 2.

For the purposes of this invention, and as is understood in the art, the term “moiety” means a chemical functional group when it is part of a larger compound, for example when part of a compound of formula (I). For example, the term “polyamine moiety” refers to the polyamine(s) portion or functionality of formula (I). Likewise the term “additive moiety” refers to the portion of formula (I) with additive functionality. Additive functionality means for example antioxidant, ultraviolet light absorber, light stabilizer, process stabilizer, etc., functionality.

The chemical linking group L may for example be any divalent linking group. Linking groups are for example esters, amides, and other common divalent groups, for example —OCO—, —COO—, —O—, —CONH—, —CONR—, —NHCO—, —NRCO—, —CO—, —NH—, —NR—, —S—, —SO—, SO₂—, —CSO—,

### —COS—, —CSS—,

where R is a hydrocarbyl group.

Linking groups L may also be a divalent hydrocarbylene group that comprises one of the above ester, amide, etc., groups.

The term “hydrocarbyl group” broadly refers to a monovalent hydrocarbon group in which the valency is derived by abstraction of a hydrogen from a carbon atom. Hydrocarbyl includes for example aliphatics (straight and branched chain), cycloaliphatics, aromatics and mixed groups such as aralkyl, alkylaryl, alkynyl, cycloalkynyl. Hydrocarbyl includes such groups as alkyl, cycloalkyl, aryl, aralkyl, alkylaryl, alkenyl, and cycloalkenyl. A hydrocarbyl may optionally be interrupted by carbonyl, carboxyl, amino, amido, thio, sulfoxide, sulfonyl and ether groups and/or may optionally be substituted by hydroxy, amino, amido, carboxyl and thio groups.

The term “hydrocarbylene” broadly refers to any divalent hydrocarbon in which both valencies derive by abstraction of hydrogens from carbon atoms. Included within the definition of hydrocarbylene are the same groups as indicated herein for hydrocarbyl, with of course, the extra valency, for example alkylene, alkenylene, arylene, alkylaryl, etc.

A hydrocarbylene as defined herein may also be any polymeric or oligomeric backbone known in the art as part of polymeric or oligomeric polymer additives. For example triazine-containing polymeric backbones that are part of commercial hindered amine compounds, for example Chimassorb® 944.

To prepare a compound or polymer of formula (I), reactive additive compounds of formula (a) are reacted with either a) a fully formed polyamine polymer or copolymer, b) a partially formed polyamine polymer or copolymer, or c) any component of a polyamine polymer or copolymer prior to its incorporation into a polyamine polymer or copolymer. The term “component” means monomer or polymer or copolymer units employed to prepare a polyamine polymer or copolymer.

Reactive additive compounds of the present invention are compounds comprised of at least one additive moiety. Reactive compounds of formula (a) comprise at least one suitable reactive functional group and/or at least one hydrocarbylene comprising at least one suitable reactive functional group:

(additive moiety)ₚ-(G)_{q} (a)

The variables p and q are independently greater than or equal to 1,

G is independently a reactive functional group (RFG) or a hydrocarbylene comprising at least one reactive functional group.

The polymer additive compounds of formula (a) contain additive functional moieties selected for example from the group consisting of antioxidant, ultraviolet light absorber, hindered amine light stabilizer, hydroxylamine stabilizer, nitrone stabilizer, amine oxide stabilizer, benzofuranone stabilizer and organic phosphorus stabilizer moieties.

The reactive functional group (RFG) may be, for example, —OH, —NHR, —NH₂, —SH, —SO₂H, —CO₂H, —CO₂R, —COX, —CSOH, —COSH, —CS₂H, —NCO, epoxy, epoxy ether, epoxy ester or X,

wherein X is Cl, Br or I and R is a hydrocarbyl group.

The additive moieties are for example chemical structural groups comprising additive functional structural groups selected from the group consisting of

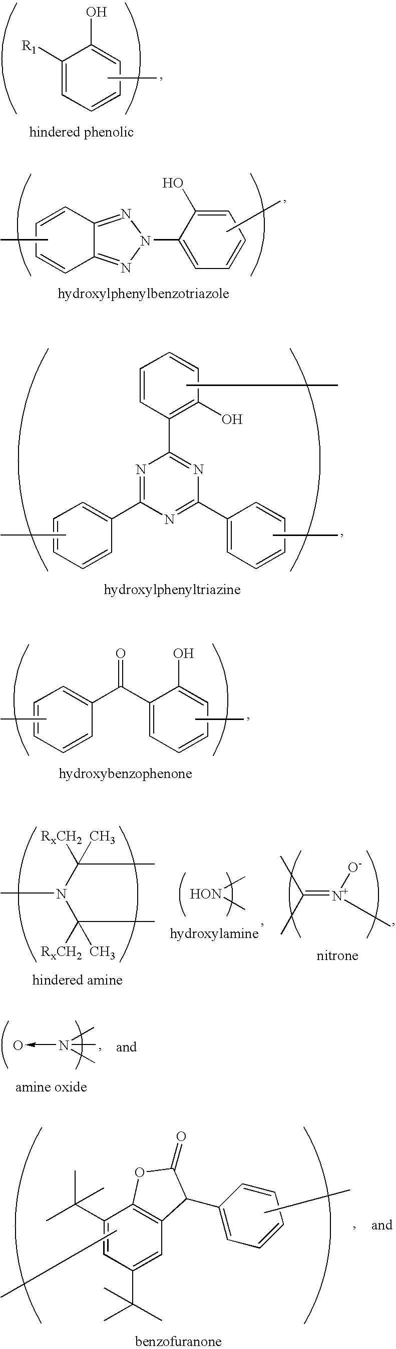

wherein at least one of the open bonds of the moieties is bound directly to a group G,
R₁ is a sterically bulky group, for example a group selected from the group consisting of tert-butyl, α-methylbenzyl, α,α-dimethyl-benzyl(cumyl), α-methylcyclohexyl, cyclopentyl, benzyl and tert-octyl, and
Rₓ is hydrogen or methyl.

The remaining open bonds are bound to groups known to those skilled in the art so that the additive moiety has its known additive function, for example hydrogen or a hydrocarbyl group or a hydrocarbylene group. It is possible for more than one hydrocarbylene to be bound to another to form a cyclic structure.

The reactive functional group, RFG, of the group G of the compound of formula (a) reacts with a reactive site on a polyamine polymer or copolymer, a partially formed polyamine polymer or copolymer, or a component thereof. The linking group L of formula (I) is formed by this reaction.

In many instances the present compounds of the formula (a) are disclosed and known to those skilled in the art of polymer stabilization. Known compounds without any reactive functional group may also be modified to have a reactive functional group; or a known compound with a reactive functional group may be modified to have a reactive functional group of a different reactive functionality. For example, a compound of formula (a) where a polymer additive moiety is attached to a group G comprising a reactive functional group such as an electrophilic ester, the ester may be reduced to be a nucleophilic alcohol.

That is, the polymer additives of formula (a) of the present invention are known in the art or are known compounds that may be modified by known methods to be of formula (a).

Specific examples of polymer additives of formula (a) are

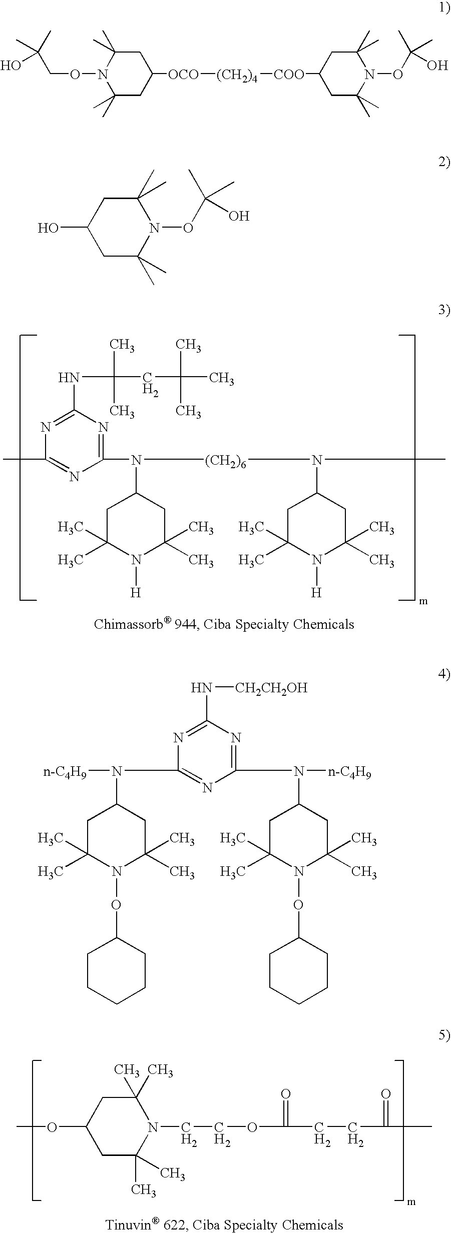

and other hindered amines or modifiable hindered amines,

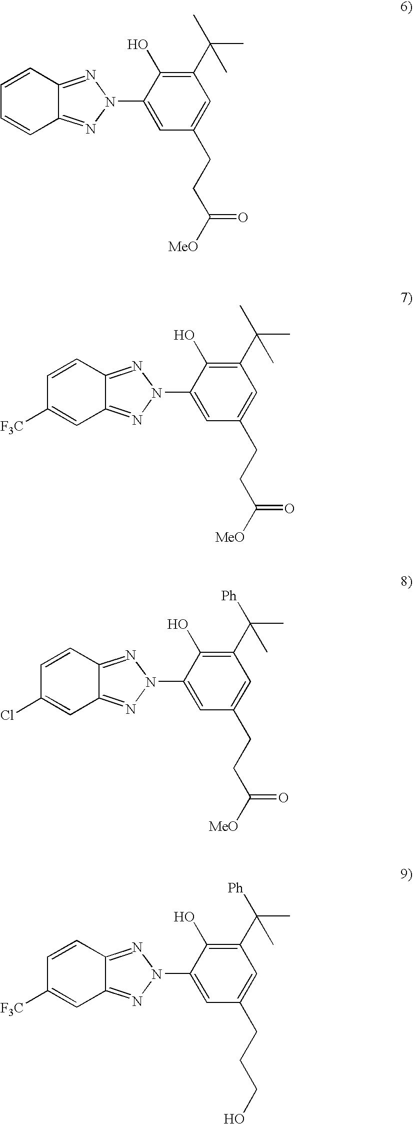

and other hydroxyphenylbenzotriazoles or modifiable hydroxyphenylbenzotriazoles,

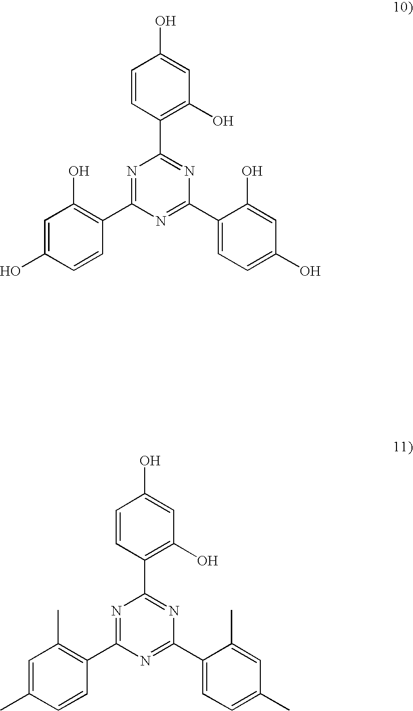

and other hydroxyphenyltriazines or modifiable triazines.

It can seen from the above structures of formulae 1)-11), that the present variables p and q may truly be independent of each other. Therefore, variables x and y in present compounds of formula (I) are also truly independent of each other.

For example, in the compound of formula 1), with a reactive hydroxyl, p is 1 and q is 2. In the compound of formula 1), the additive moiety is

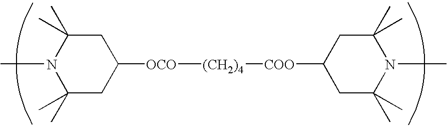

and the group G is a hydrocarbyl group comprising the reactive functional group hydroxyl:

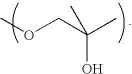

In formula 2), p is 1 and q is 2. The additive moiety is

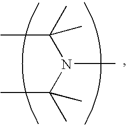

and there are two different hydrocarbyl groups G comprising reactive functional groups (hydroxyls):

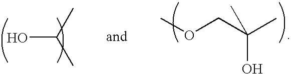

In oligomeric formula 3), with a reactive amine end group, p=2 times m, the group G may be considered the oligomeric backbone comprising the hindered amine moieties and therefore q is 1.

In tris-resorcinol triazine 10), with 1, 2, 3, 4 or 5 reactive hydroxyl groups, p is 1 and q is 5, and each G is hydroxyl. It can be seen that if a tris-resorcinol triazine of formula 10) is attached to a polyamine moiety 1, 2, 3, 4 or 5 times, that x and y are independent of each other in compounds of formula (I).

The additive functional structural groups that are sub-structures (a part thereof) of the additive moieties of the present invention are disclosed in many U.S. patents and are known to those skilled in the art. They are the functional portions of the additives disclosed and known in the art. For example, the chromophore of a known ultraviolet light absorber (UVA) is the primary functional portion (functional structural group) of the UVA molecule.

For example, the hydroxyphenylbenzotriazole functional structural groups are disclosed for example in U.S. Pat. Nos. 3,004,896; 3,055,896; 3,072,585; 3,074,910; 3,189,615; 3,218,332; 3,230,194; 4,127,586; 4,226,763; 4,275,004; 4,278,589; 4,315,848; 4,347,180; 4,383,863; 4,675,352; 4,681,905, 4,853,471; 5,268,450; 5,278,314; 5,280,124; 5,319,091; 5,410,071; 5,436,349; 5,516,914; 5,554,760; 5,563,242; 5,574,166; 5,607,987, 5,977,219 and 6,166,218, the relevant parts of which are hereby incorporated by reference.

The hydroxyphenyltriazine functional structural groups are disclosed for example in U.S. Pat. Nos. 3,843,371; 4,619,956; 4,740,542; 5,096,489; 5,106,891; 5,298,067; 5,300,414; 5,354,794; 5,461,151; 5,476,937; 5,489,503; 5,543,518; 5,556,973; 5,597,854; 5,681,955; 5,726,309; 5,942,626; 5,959,008; 5,998,116 and 6,013,704, and U.S. application Ser. No. 09/383,163, the relevant parts of which are hereby incorporated by reference.

The hindered amine functional structural groups are disclosed for example in U.S. application Ser. Nos. 09/257,711, 09/505,529 and 09/794,710, and U.S. Pat. Nos. 5,204,473, 5,096,950, 5,004,770, 5,844,026, 6,046,304, 6,166,212, 6,117,995 and 6,221,937, the relevant parts of which are hereby incorporated by reference. The amine of the hindered amine may be substituted by groups known in the art, for example methyl, hydrogen, acyl, or alkoxy or cycloalkoxy.

Hydroxylamine functional structural groups are disclosed for example in U.S. Pat. Nos. 4,590,231, 4,668,721, 4,782,105 and 4,876,300, 4,649,221, 4,691,015, 4,703,073, 4,612,393, 4,696,964, 4,720,517 and 4,757,102, 4,831,134, 5,006,577, 5,019,285, 5,064,883, 5,185,448 and 5,235,056, 4,666,962, 4,666,963, 4,678,826, 4,753,972, 4,757,102, 4,760,179, 4,929,657, 5,057,563, 5,021,479, 5,045,583 and 5,185,448, the relevant parts of which are hereby incorporated by reference.

Amine oxide functional structural groups are disclosed for example in U.S. Pat. Nos. 5,081,300, 5,162,408, 5,844,029, 5,880,191 and 5,922,794, the relevant parts of which are hereby incorporated by reference.

Nitrone functional structural groups are disclosed for example in U.S. Pat. No. 4,898,901, the relevant parts of which are hereby incorporated by reference.

Benzofuranone functional structural groups are disclosed for example in U.S. Pat. Nos. 4,325,863; 4,338,244; 5,175,312; 5,216,052; 5,252,643; 5,369,159; 5,488,117; 5,356,966; 5,367,008; 5,428,162; 5,428,177; 5,614,572; 5,883,165 or 5,516,920, all incorporated herein by reference.

In another embodiment of the instant invention, the sunless tanning agent of component i) is present in the compositions of the invention in concentrations ranging from 0.01% to 50% by weight based on the total weight of the composition.

In another embodiment of the instant invention, the sunless tanning agent of component i) is present in the compositions of the invention in concentrations ranging from 0.1% to 20% by weight based on the total weight of the composition.

In another embodiment of the instant invention, the sunless tanning agent of component i) is present in the compositions of the invention in concentrations ranging from 0.5% to 10% by weight based on the total weight of the composition.

In another embodiment of the instant invention, the substituted polyamine compounds of formula (I) in component ii) are present in the compositions of the invention in concentrations ranging from 0.01% to 50% by weight based on the total weight of the composition.

In another embodiment of the instant invention, the substituted polyamine compounds of formula (I) in component ii) are present in the compositions of the invention in concentrations ranging from 0.1% to 20% by weight based on the total weight of the composition.

In another embodiment of the instant invention, the substituted polyamine compounds of formula (I) in component ii) are present in the compositions of the invention in concentrations ranging from 0.5% to 10% by weight based on the total weight of the composition.

DHA may be used in free form and/or encapsulated, for example in lipid vesicles such as liposomes, described especially in WO 97/25970. In addition, a DHA precursor may be used as described especially in US 2003/0185769.

These sunless tanning agents may be combined with at least one synthetic or natural direct dye and/or at least one indole derivative, for instance those described in patents EP 425 324 and EP 456 545.

These self-tanning agents may also be combined with other synthetic or natural skin-coloring agents.

For the purposes of the present invention, the expression “skin-coloring agent” will mean any compound with particular affinity for the skin, making it possible to give the skin a long-lasting, non-covering (i.e. not having a tendency to opacify the skin) coloration and which is not removed either with water or using a solvent, and which is resistant both to rubbing and to washing with a solution containing surfactants. Such a long-lasting coloration is thus distinguished from the superficial and transient coloration provided, for example, by a makeup pigment.

The additional coloring agents may also be chosen, for example, from plant extracts such as, for example, “insoluble” extracts of red woods of the genus Pterocarpus and of the genus Baphia, for instance Pterocarpus santalinus, Pterocarpus osun, Pterocarpus soyauxii, Pterocarpus erinaceus, Pterocarpus indicus or Baphia nitida, for instance those described in patent application EP 971 683.

The coloring agents may also be iron oxide nanopigments, the mean size of the elementary particles of which is less than 100 nm, such as those described in patent application EP 966 953.

The sunless tanning compositions in accordance with the invention may be in the form of creams, milks, gels, cream-gels, oil-in-water emulsions, vesicular dispersions, fluid lotions, in particular vaporizable fluid lotions, or any other form generally used in cosmetics, in particular those usually suitable for sunless tanning cosmetic compositions.

The compositions in accordance with the present invention may also comprise cosmetically acceptable adjuvants chosen especially from fatty substances, organic solvents, ionic or nonionic thickeners, softeners, antioxidants, free-radical scavengers, opacifiers, stabilizers, emollients, silicones, .alpha.-hydroxy acids, antifoams, moisturizers, vitamins, insect repellants, substance p antagonists, anti-inflammatories, fragrances, preserving agents, surfactants, fillers, polymers other than those of the invention, propellants, acidifying or basifying agents, colorants or any other ingredient usually used in cosmetics and/or dermatology, in particular for the manufacture of self-tanning compositions in the form of emulsions.

The fatty substances may consist of an oil or a wax or mixtures thereof. The term “oil” means a compound that is liquid at room temperature. The term “wax” means a compound that is solid or substantially solid at room temperature, and whose melting point is generally greater than 35 C.

Oils that may be mentioned include mineral oils (paraffin); plant oils (sweet almond oil, macademia oil, blackcurrant pip oil, jojoba oil); synthetic oils, for instance perhydrosqualene, fatty alcohols, fatty acids or fatty esters (for instance the C12-C15 alkyl benzoate sold under the trade name “Finsolv TN” by the company Finetex), octyl palmitate, isopropyl lanolate, triglycerides, including those of capric/caprylic acids, oxyethylenated or oxypropylenated fatty esters and ethers; silicone oils (cyclomethicone, polydimethylsiloxanes or PDMS) or fluoro oils; polyalkylenes, and mixtures thereof.

Waxy compounds that may be mentioned include paraffin, carnauba wax, beeswax and hydrogenated castor oil.

Among the organic solvents that may be mentioned are lower alcohols and polyols containing not more than 8 carbon atoms.

The thickeners may be chosen especially from crosslinked polyacrylic acids, and modified or unmodified guar gums and celluloses, such as hydroxypropyl guar gum, methylhydroxyethylcellulose and hydroxypropylmethylcellulose or mixtures thereof.

The compositions in accordance with the invention may also comprise at least one organic photoprotective agent and/or at least one inorganic photoprotective agent that is active in the UVA and/or UVB range (absorbent) and are water-soluble or liposoluble or even insoluble in the cosmetic solvents commonly used. These organic and inorganic photoprotective agents are not covalently bound to the substituted polyamine of formula (I) of component ii).

The organic photoprotective agents are especially chosen from anthranilates; cinnamic derivatives; dibenzoylmethane derivatives; salicylic derivatives; camphor derivatives; triazine derivatives such as those described in U.S. Pat. Nos. 4,367,390, 4,724,137, EP 863 145, EP 517 104, EP 570 838, EP 796 851, EP 775 698, EP 878 469, EP 933 376, EP 506 691, EP 507 692, EP 790 243 and EP 944 624; benzophenone derivatives; .beta.,.beta.-diphenylacrylate derivatives; benzotriazole derivatives; benzalmalonate derivatives; benzimidazole derivatives; imidazolines; bis-benzazolyl derivatives as described in EP 669 323 and U.S. Pat. No. 2,463,264; p-aminobenzoic acid (PABA) derivatives; methylenebis(hydroxyphenyl)benzotriazole derivatives as described in U.S. Pat. Nos. 5,237,071, 5,166,355, GB 2 303 549, DE 197 26 184 and EP 893 119; screening polymers and screening silicones such as those described especially in patent application WO 93/04665; dimers derived from .alpha.-alkylstyrene, such as those described in patent application DE 198 55 649, 4,4-diarylbutadienes such as those described in patent applications EP 0 967 200 and DE 197 55 649, and mixtures thereof.

As examples of UV-A-active and/or UV-B-active organic photoprotective agents, mention may be made of the following, denoted hereinbelow under their INCI name:

Para-Aminobenzoic Acid Derivatives PABA, Ethyl PABA, Ethyl dihydroxypropyl PABA, Ethylhexyl dimethyl PABA sold in particular under the name “Escalol 507” by ISP, Glyceryl PABA, and PEG-25 PABA sold under the name “Uvinul P25” by BASF.

Salicylic derivatives: Homosalate sold under the name “Eusolex HMS” by RonalEM Industries, Ethylhexyl salicylate sold under the name “Neo Heliopan OS” by Haarmann and Reimer, Dipropylene glycol salicylate sold under the name “Dipsal” by Scher, and TEA salicylate sold under the name “Neo Heliopan TS” by Haarmann and Reimer.

Dibenzoylmethane Derivatives Butyl methoxydibenzoylmethane sold in particular under the trade name “Parsol 1789” by Hoffmann LaRoche, and Isopropyldibenzoylmethane.

Cinnamic Derivatives Ethylhexyl methoxycinnamate sold in particular under the trade name “Parsol MCX” by Hoffmann LaRoche, Isopropyl methoxycinnamate, Isoamyl methoxycinnamate sold under the trade name “Neo Heliopan E 1000” by Haarmann and Reimer, Cinoxate, DEA methoxycinnamate, and Glyceryl ethylhexanoate dimethoxycinnamate.

Alpha,alpha.′-Diphenol Acrylate Derivatives Octocrylene sold in particular under the trade name “Uvinul N539” by BASF, and Etocrylene sold in particular under the trade name “Uvinal N35” by BASF.

Benzophenone Derivatives Benzophenone-1 sold under the trade name “Uvinul 400” by BASF, Benzophenone-2 sold under the trade name “Uvinul D50” by BASF, Benzophenone-3 or Oxybenzone sold under the trade name “Uvinul M40” by BASF, Benzophenone-4 sold under the trade name “Uvinul MS40” by BASF, Benzophenone-5, Benzophenone-6 sold under the trade name “Helisorb 11” by Norquay, Benzophenone-8 sold under the trade name “Spectra-Sorb UV-24” by American Cyanamid, Benzophenone-9 sold under the trade name “Uvinul DS-49” by BASF, and Benzophenone-12.

Benzylidenecamphor Derivatives 3-Benzylidenecamphor manufactured under the name “Mexoryl SD” by Chimex, 4-Methylbenzylidenecamphor sold under the name “Eusolex 6300” by Merck, Benzylidenecamphorsulphonic acid manufactured under the name “Mexoryl SO” by Chimex, Camphor benzalkonium methosulfate manufactured under the name “Mexoryl SO” by Chimex, Terephthalylidenedicamphorsulphonic acid manufactured under the name “Mexoryl SX” by Chimex, and Polyacrylamidomethylbenzylidenecamphor manufactured under the name “Mexoryl SW” by Chimex.

Benzimidazole Derivatives Phenylbenzimidazolesulphonic acid sold in particular under the trade name “Eusolex 232” by Merck, and Benzimidazilate sold under the trade name “Neo Heliopan AP” by Haarmann and Reimer.

Triazine Derivatives Anisotriazine sold under the trade name “Tinosorb 5” by Ciba Specialty Chemicals, Ethylhexyltriazone sold in particular under the trade name “Uvinul T150” by BASF, Diethylhexylbutamidotriazone sold under the trade name “Uvasorb HEB” by Sigma 3V, and 2,4,6-Tris(diisobutyl 4′-aminobenzalmalonate) s-triazine.

Benzotriazole Derivatives Drometrizole trisiloxane sold under the name “Silatrizole” by Rhodia Chimie, and Methylenebis(benzotriazolyl)tetramethylbutylphenol sold in solid form under the trade name “MIXXIM BB/100” by Fairmount Chemical, or in micronized form as an aqueous dispersion under the trade name “Tinosorb M” by Ciba Specialty Chemicals.

Anthranilic Derivatives Menthyl anthranilate sold under the trade name “Neo Heliopan MA” by Haarmann and Reimer.

Imidazoline Derivatives Ethylhexyldimethoxybenzylidenedioxoimidazoline propionate.

Benzalmalonate Derivatives Polyorganosiloxane containing benzalmalonate functions, sold under the trade name “Parsol SLX” by Hoffmann LaRoche and mixtures thereof.

The organic photoprotective agents that are more particularly preferred are chosen from the following compounds: Ethylhexyl salicylate, Butyl methoxydibenzoylmethane, Ethylhexyl methoxycinnamate, Octocrylene, Phenylbenzimidazolesulphonic acid, Terephthalylidenedicamphorsulphonic acid, Benzophenone-3, Benzophenone-4, Benzophenone-5,4-Methylbenzylidenecamphor, Benzimidazilate, Anisotriazine, Ethylhexyltriazone, Diethylhexylbutamidotriazone, Methylenebis(benzotriazolyl)tetramethylbutylphenol, Drometrizole trisiloxane, 2,4,6-tris(diisobutyl 4′-aminobenzalmalonate)-s-triazine, and mixtures thereof.

The inorganic photoprotective agents are chosen from pigments or nanopigments (mean size of the primary particles: generally between 5 nm and 100 nm and preferably between 10 nm and 50 nm) of coated or uncoated metal oxides such as, for example, nanopigments of titanium oxide (amorphous or crystallized in rutile and/or anatase form), of iron oxide, of zinc oxide, of zirconium oxide and of cerium oxide, and mixtures thereof. Standard coating agents are, moreover, alumina and/or aluminium stearate. Such coated or uncoated metal oxide nanopigments are described in particular in patent applications EP 518 772 and EP 518 773.

The photoprotective agents are generally present in the compositions according to the invention in proportions ranging from 0.1% to 20% by weight relative to the total weight of the composition, and preferably ranging from 0.2% to 15% by weight relative to the total weight of the composition.

Needless to say, the person skilled in the art will take care to select the abovementioned optional additional compound(s) and/or the amounts thereof such that the advantageous properties intrinsic to the combination in accordance with the invention are not, or not substantially, adversely affected by the envisaged addition(s).

The compositions of the invention may be prepared according to techniques that are well known to those skilled in the art, in particular those intended for preparing emulsions of oil-in-water or water-in-oil type.

This composition may be in the form of a simple or complex emulsion (O/W, W/O, O/W/O or W/O/W emulsion) such as a cream, a milk or in the form of a gel or a cream-gel, in the form of a lotion, a powder or a solid tube, and may optionally be packaged as an aerosol and be in the form of a mousse or a spray.

Preferably, the compositions according to the invention are in the form of an oil-in-water or water-in-oil emulsion.

When it is an emulsion, the aqueous phase of this emulsion may comprise a nonionic vesicular dispersion prepared according to known processes (Bangham, Standish and Watkins, J. Mol. Biol. 1965, 13, 238; FR 2 315 991 and FR 2 416 008).

The invention also relates to a cosmetic treatment process for artificially tanning and/or browning the skin, characterized in that it consists in applying to the skin an effective amount of a cosmetic composition as defined above.

The invention also relates to the use of a substituted polyamine of formula (I) of component ii) as defined above with the aim of improving the coloration and/or stability of a self-tanning agent such as those defined above, contained in a cosmetic composition for artificially tanning and/or browning the skin.

The actual active ingredient and the actual minimum effective amount will be determined by the actual product/application in which the cosmetic composition is to be used.

The following examples describe certain embodiments of this invention, but the invention is not limited thereto. It should be understood that numerous changes to the disclosed embodiments could be made in accordance with the disclosure herein without departing from the spirit or scope of the invention. These examples are therefore not meant to limit the scope of the invention. Rather, the scope of the invention is to be determined only by the appended claims and their equivalents. In these examples all parts given are by weight unless otherwise indicated.

The following examples illustrate the invention.

### EXAMPLE 1

### Condensation Polymer from L-Lysine and 2-[2′-Hydroxy-3′-t-butyl-5′-(2-methoxycarbonyl ethyl)phenyl]-benzotriazole (Benzotriazole UV Absorber)

Into a 250 ml three-neck round bottom flask is placed L-Lysine (100 g, 0.54 mol, Aldrich) and NaOH (43.8 g, 0.54 mol, 50% assay) is added over 20 minutes with stirring which becomes a milky white mixture. The reaction temperature is initially at 115 C but increased to 150 C. At this temperature, phosphoric acid (3.8 g, 85%, 33.0 mmol) is added to the mixture and the temperature is increased to 170 C under vacuum. The water in the reaction mixture is azeotroped over along with some from the reaction mass as the temperature reaches 170 C. The reaction mixture is heated and stirred for two hours under increasing vacuum. After which, 2-[2′-Hydroxy-3′-t-butyl-5′-(2-methoxycarbonyl ethyl)phenyl]-benzotriazole (BZT) (42.1 g, 0.117 mol, Ciba) is added to the mixture with stirring; the temperature drops to 140 C. The reaction temperature is gradually increased to 195 C and is held there for 2 hours. The progress of the condensation is monitored by the amount of water/methanol collected and by gas chromatography. After two hours, 3.4 g of distillate (91% of the theoretical weight of methanol) is collected and gas chromatography indicates that <0.2 wt % of the starting BTZ remains unreacted. A brown solid is obtained (142.3 g, 92.3% yield) with a melting point of 142 C. A clear light brown gel is formed upon dissolving in water and adjusting pH to 4-5.

### EXAMPLE 2

### Condensation Polymer from L-Lysine and 2-[2′-Hydroxy-3′-t-butyl-5′-(2-methoxycarbonyl ethyl)phenyl]-benzotriazole (Benzotriazole UV Absorber)

Into a 250 ml three-neck round bottom flask is placed L-Lysine (100 g, 0.54 mol, Aldrich) and NaOH (21.9 g, 0.27 mol, 50% assay) is added over 20 minutes with stirring which becomes a milky white mixture. The reaction temperature is initially at 115 C but increased to 150 C. At this temperature, phosphoric acid (1.9 g, 85%, 16.5 mmol) is added to the mixture and the temperature is increased to 170 C under vacuum. The water in the reaction mixture is azeotroped over along with some from the reaction mass as the temperature reaches 170 C. The reaction mixture is heated and stirred for two hours under increasing vacuum. After which, 2-[2′-Hydroxy-3′-t-butyl-5′-(2-methoxycarbonyl ethyl)phenyl]-benzotriazole (BZT) (12.3 g, 0.034 mol, Ciba) is added to the mixture with stirring; the temperature drops to 140 C. The reaction temperature is gradually increased to 195 C and is held there for 2 hours. The progress of the condensation is monitored by the amount of water/methanol collected and by gas chromatography. After two hours, gas chromatography indicates that the reaction is complete. A brown solid is obtained (65 g, 94.1% yield) with a melting point of ????C. A cloudy gel is formed upon dissolving in water and adjusting pH to 4-5 with 22.8 weight percent of polymer solids.

### EXAMPLE 3

### Condensation Polymer from L-Lysine and Methyl 3,5-di-tert-butyl-4-hydroxyhydrocinnamate (Phenolic Antioxidant)

Into a 250 ml three-neck round bottom flask is placed L-Lysine (50.0 g, 0.27 mol, Aldrich) and NaOH (21.9 g, 0.27 mol, 50% assay) is added over 20 minutes with stirring which becomes a milky white mixture. The reaction temperature is initially at 115 C but increased to 150 C. At this temperature, phosphoric acid (1.9 g, 85%, 16.5 mmol) is added slowly to the mixture and heated to 170 C. under vacuum. The water in the reaction mixture is azeotroped over along with some from the reaction mass as the temperature reaches 170 C. The reaction mixture is heated and stirred for two hours under increasing vacuum. After which, methyl 3,5-di-tert-butyl-4-hydroxyhydrocinnamate (25 g, 0.05 mol, Ciba) is added to the mixture with stirring; the temperature drops to 140 C. The reaction temperature is gradually increased to 195 C and is held there for 2 hours. The progress of the condensation is monitored by gas chromatography. After two hours, gas chromatography indicates that <1.0 wt % of the starting methyl ester is remaining. A brown solid is obtained (67.4 g, 86.1%) with a melting point of 82 C. A clear dark brown liquid is formed upon dissolving in water and adjusting pH to 4-5.

Table 1 summarizes the properties of the above polymer and others prepared analogously.

TABLE 1

| | Condensed | |
|---|---|---|
| | component | Condensed component |
| Example | 1 (Mol ratio¹) | 1 (Mol ratio¹) |
| 4 | BZT (0.1) | Antioxidant (0.1) |
| 5 | BZT (0.2) | Antioxidant (0.2) |
| 6 | BZT (0.4) | Antioxidant (0.4) |
| 7 | BZT (0.5) | Antioxidant (0.1) |
| 8 | BZT (0.5) | Antioxidant (0.3) |

¹Based on L-Lysine content

### EXAMPLE 9

### Condensation Polymer from L-Lysine and 2-[2′-Hydroxy-3′-t-butyl-5′-(2-methoxycarbonyl ethyl)phenyl]-benzotriazole (Benzotriazole UV Absorber) and Methyl Undecanoate (Fatty Acid Ester)

Into a 250 ml three-neck round bottom flask is placed L-Lysine (100 g, 0.54 mol, Aldrich) and NaOH (43.8 g, 0.54 mol, 50% assay) is added over 20 minutes with stirring which becomes a milky white mixture. The reaction temperature is initially at 115 C but increased to 150 C. At this temperature, phosphoric acid (3.8 g, 85%, 33.0 mmol) is added to the mixture and heated to a 170 C under vacuum. The water in the reaction mixture is azeotroped over along with some from the reaction mass as the temperature reaches 170 C. The reaction mixture is heated and stirred for two hours under increasing vacuum. After which, 2-[2′-Hydroxy-3′-t-butyl-5′-(2-methoxycarbonyl ethyl)phenyl]-benzotriazole (BZT) (42.1 g, 0.117 mol, Ciba) and methyl undecanoate (11.0 g, 54.9 mmol, Aldrich) are added to the mixture with stirring; the temperature drops to 140 C. The reaction temperature is gradually increased to 195 C and is held there for 2 hours. The progress of the condensation is monitored by gas chromatography. After two hours, the reaction is complete as determined by gas chromatography which indicates that <1.0 wt % of the starting BTZ and methyl undecanoate is remaining. A brown solid is obtained.

Table 2 summarizes the properties of the above polymer and others prepared analogously.

TABLE 2

| | Condensed | Condensed | Other Condensed |
|---|---|---|---|
| | component | component | component 1 |
| Example | 1 (Mol ratio¹) | 1 (Mol ratio¹) | (Mol ratio¹) |
| 10 | BZT (0.1) | Antioxidant (0.1) | Lauric acid (0.1) |
| 11 | BZT (0.2) | Antioxidant (0.2) | Lauric acid (0.2) |
| 12 | BZT (0.4) | Antioxidant (0.4) | Lauric acid (0.1) |
| 13 | BZT (0.5) | Antioxidant (0.1) | Lauric acid (0.2) |
| 14 | BZT (0.5) | Antioxidant (0.3) | Lauric acid (0.1) |
| 15 | BZT (0.1) | Antioxidant (0.1) | Stearic acid (0.1) |
| 16 | BZT (0.2) | Antioxidant (0.2) | Stearic acid (0.2) |
| 17 | BZT (0.4) | Antioxidant (0.4) | Stearic acid (0.1) |
| 18 | BZT (0.5) | Antioxidant (0.1) | Stearic acid (0.2) |
| 19 | BZT (0.5) | Antioxidant (0.3) | Stearic acid (0.1) |

¹Based on L-Lysine content

### EXAMPLE 20

### Condensation Polymer from L-Lysine and 2-[2′-Hydroxy-3′-t-butyl-5′-(2-methoxycarbonyl ethyl)phenyl]-benzotriazole (Benzotriazole UV Absorber) grafted with a fatty acid

The solid product from Example 1 (30 g) is added to a 100 ml three-necked, round-bottomed flask equipped with stirrer, condenser and nitrogen sparge inlet, and heated to 170° C. while stirring. The solid becomes a stirrable liquid when a temperature of greater than 100 C is reached. At 100 C, NaOH (1.5 g, 12.5 mmol, 50% assay) is added over 5 minutes while heating is continued. The water in the reaction mixture is azeotroped over along with some water from the reaction as the temperature reaches 170° C. After which, undecanoyl chloride (3.4 g, 16.6 mmol) is added slowly dropwise to the mixture. The reaction temperature is gradually increased to 195° C. and is held there for 2 hours. The progress of the reaction is monitored by chloride titration and gas chromatography. After two hours, the reaction is complete. A brown solid is obtained.

Table 3 summarizes the properties of the above polymer and others prepared analogously.

TABLE 3

| | Condensed | Condensed | Other Grafted |
|---|---|---|---|
| | component | component | component |
| Example | 1 (Mol ratio¹) | 1 (Mol ratio¹) | 1 (Mol ratio¹) |
| 21 | BZT (0.1) | Antioxidant (0.1) | Lauroyl chloride (0.1) |
| 22 | BZT (0.2) | Antioxidant (0.2) | Lauroyl chloride (0.2) |
| 23 | BZT (0.4) | Antioxidant (0.4) | Lauroyl chloride (0.1) |
| 24 | BZT (0.5) | Antioxidant (0.1) | Lauroyl chloride (0.2) |
| 25 | BZT (0.5) | Antioxidant (0.3) | Lauroyl chloride (0.1) |
| 26 | BZT (0.1) | Antioxidant (0.1) | Stearoyl chloride (0.1) |
| 27 | BZT (0.2) | Antioxidant (0.2) | Stearoyl chloride (0.2) |
| 28 | BZT (0.4) | Antioxidant (0.4) | Stearoyl chloride (0.1) |
| 29 | BZT (0.5) | Antioxidant (0.1) | Stearoyl chloride (0.2) |
| 30 | BZT (0.5) | Antioxidant (0.3) | Stearoyl chloride (0.1) |

¹Based on L-Lysine content

### EXAMPLE 31

### Condensation Polymer from L-Lysine and 2-[2′-Hydroxy-3′-t-butyl-5′-(2-methoxycarbonyl ethyl)phenyl]-benzotriazole (Benzotriazole UV Absorber) further reacted with an epoxide containing compound

The solid product from Example 1 (30 g) is added to a 100 ml three-necked, round-bottomed flask equipped with stirrer, condenser and nitrogen sparge inlet, and heated to 70 C. while stirring. The solid is dissolved in water and the pH of the mixture was adjusted with NaOH (1.5 g, 18.8 mmol, 50% assay) to obtain a pH between 8-10. The reaction mixture is stirred and heated to 70 C. Quab 342 (14.8 g, 16.4 mmol) is added dropwise over 10 minutes to the mixture. The reaction temperature is increased to 80 C and is held there for 2 hours. The progress of the condensation is monitored by chloride titration and liquid chromatography. After two hours, the reaction is complete as determined by chloride titration.

Table 4 summarizes the properties of the above polymer and others prepared analogously.

TABLE 4

| | Condensed | Condensed | Grafted |
|---|---|---|---|
| | component | component | component 2 |
| Example | 1 (Mol ratio¹) | 1 (Mol ratio¹) | (Mol ratio¹) |
| 32 | BZT (0.1) | Antioxidant (0.1) | Quab 342 (0.3) |
| 33 | BZT (0.2) | Antioxidant (0.2) | Quab 342 (0.5) |
| 34 | BZT (0.4) | Antioxidant (0.4) | Quab 342 (0.1) |
| 35 | BZT (0.5) | Antioxidant (0.1) | Quab 342 (0.3) |
| 36 | BZT (0.5) | Antioxidant (0.3) | Quab 342 (0.1) |
| 37 | BZT (0.1) | Antioxidant (0.1) | Quab 426 (0.5) |
| 38 | BZT (0.2) | Antioxidant (0.2) | Quab 426 (0.3) |
| 39 | BZT (0.4) | Antioxidant (0.4) | Quab 426 (0.1) |
| 40 | BZT (0.5) | Antioxidant (0.1) | Quab 426 (0.3) |
| 41 | BZT (0.5) | Antioxidant (0.3) | Quab 426 (0.1) |
| 42 | BZT (0.1) | Antioxidant (0.1) | E-dodecane (0.3) |
| 43 | BZT (0.2) | Antioxidant (0.2) | E-dodecane (0.2) |
| 44 | BZT (0.4) | Antioxidant (0.4) | E-dodecane (0.1) |
| 45 | BZT (0.5) | Antioxidant (0.1) | E-dodecane (0.2) |
| 46 | BZT (0.5) | Antioxidant (0.3) | E-dodecane (0.1) |

¹Based on L-Lysine content
Quab 342 = 3-chloro-2-hydroxypropyl-dimethyldodecylammonium chloride, Degussa
Quab 426 = 3-chloro-2-hydroxypropyl-dimethyloctadecylammonium chloride, Degussa
E-Dodecane = 1,2-epoxydodecane

### EXAMPLE 47

### Condensation Polymer from L-Lysine and 2-[2′-Hydroxy-3′-t-butyl-5′-(2-methoxycarbonyl ethyl)phenyl]-benzotriazole (Benzotriazole UV Absorber) further reacted with epichlorohydrin

The formulated product (7.7% solids) from Example 1 (15.2 g) and 20.0 g of distilled water are added to a 100 ml three-necked, round-bottomed flask equipped with stirrer, condenser and nitrogen sparge inlet, and heated to 70 C. while stirring. NaOH (0.5 g, 6.3 mmol, 50% assay) is then added over 5 minutes. Epichlorohydrin (0.11 g, 1.1 mmol) is added to the mixture. The contents are stirred at 70 C for 2 hours. After which, the reaction of the epichlorohydrin is complete as determined by gas chromatography. A clear beige liquid is obtained by dissolving in propylene glycol and water and adjusting pH to 4-5.

### EXAMPLE 48

### Condensation Polymer from L-Lysine and 2-[2′-Hydroxy-3′-t-butyl-5′-(2-methoxycarbonyl ethyl)phenyl]-benzotriazole (Benzotriazole UV Absorber) further reacted with a difunctional epoxysiloxane

The procedure of Example 47 is followed except that 1.0 g (1.1 mmol) of linear diepoxy polydimethylsiloxane (Tego 4150 from Degussa) is added instead of epichlorohydrin. A brown solid is obtained. A beige liquid is obtained by dissolving in propylene glycol and water and adjusting pH to 4-5.

### EXAMPLE 49

### Condensation Polymer from L-Lysine, glutamic acid, and 2-[2′-Hydroxy-3′-t-butyl-5′-(2-methoxycarbonyl ethyl)phenyl]-benzotriazole (Benzotriazole UV Absorber)

Into a 250 ml three-neck round bottom flask are placed L-Lysine (50.0 g, 0.27 mol) and NaOH (21.9 g, 0.27 mol, 50% assay) is added over 20 minutes with stirring to become a milky white mixture. The reaction temperature is initially at 135 C. 2-[2′-Hydroxy-3′-t-butyl-5′-(2-methoxycarbonyl ethyl)phenyl]-benzotriazole (BZT) (24.6 g, 0.068 mol) is added to the mixture with stirring. The mixture is heated to 165 C and stirred for 3 hours. The water in the reaction mixture is azeotroped over along with methanol from the reaction. Afterwards, phosphoric acid (1.9 g, 85% assay, 16.4 mmol) and glutamic acid (14.7 g, 100 mmol) are added to the mixture. The reaction mixture is heated up to 200° C. and stirred for two hours. A brown solid is obtained.

### EXAMPLE 50

### Condensation Polymer from L-Lysine, glutamic acid, and 2-[2′-Hydroxy-3′-t-butyl-5′-(2-methoxycarbonyl ethyl)phenyl]-benzotriazole (Benzotriazole UV Absorber)

The procedure of Example 49 is followed except that 12.3 g instead of 24.6 g of 2-[2′-Hydroxy-3′-t-butyl-5′-(2-methoxycarbonyl ethyl)phenyl]-benzotriazole (BZT) agent is added.

### EXAMPLE 51

### Condensation Polymer from L-Lysine, aspartic acid, and 2-[2′-Hydroxy-3′-t-butyl-5′-(2-methoxycarbonyl ethyl)phenyl]-benzotriazole (Benzotriazole UV Absorber)

The procedure of Example 49 is followed except that 13.3 g aspartic acid (100 mmol) is added instead of glutamic acid.

### EXAMPLE 52

### Testing Protocol

The test protocol described below is used to mimic the application of the sunless tanning compositions to human skin and test the color development at specified intervals of time.

The following laboratory equipment is used:

VITRO-SKIN® N-19, Foam block, Hydration Chamber, Powder Free Rubber Finger Cots and Glassless slide mounts are obtained from IMS, Inc. (70 Robinson Blvd, Orange, Conn., USA);
Water bath (#05-719-7F), Corning Hotplate Stirrer (#11-497-8A), Calfamo Compact Digital Stirrer (#14-500-7), Glycerol Aqueous Solution (#AC277366-0010) are obtained from Fisher Scientific;

### Kruss Goniometer Drop Shape Analyzer

Nicolet Avatar 370 DTGS from Thermo Electron Corporation
PerkinElmer UV/VIS Spectrophotometer Lambda 35 with Integrating Sphere Device RSA-PE 20

### ColorTec-PSM Chromameter

VITRO-SKIN (N-19) is selected as a substrate in all in vitro experiments because it effectively mimics the surface properties of human skin. It contains both optimized protein and lipid components and is designed to have topography, pH, critical surface tension and ionic strength similar to human skin. According to the manufacturer, it is used in a broad range of in vitro methods including the measurement of SPF/UVA protection factors, evaluation of the water resistance and photostability of sunscreen formulations, assessment of the performance of sunless tanning formulations, evaluation of the performance of adhesive bandages and assessment of emollient spreading.

An aqueous solution of glycerin (300 g of 14.7% by weight) is prepared and poured on the bottom of the hydration chamber. The shelves are placed in the chamber that is covered with a lid. VITRO-SKIN substrate is cut into 4 cm×4 cm pieces that are placed on the shelves in a hydration chamber and hydrated for 16-22 hours prior to the tests.

Experiments are conducted according to the methodology described in International Journal of Cosmetic Science, 2002, 24, 2-3, with following modifications: the humidity of the hydration chamber is brought about with the use of 15% glycerin solution to meet IMS recommendations; the sample size of vitro skin is 4×4 cm; the humidity of the hydration chamber for color development is controlled by 85% glycerin solution; the chromameter used in this experiment is the ColorTec-PSM; the color of the samples is measured 24 and 48 hours after being placed in chamber; and the color development chamber is kept at room temperature.

A piece of substrate is placed on a slide mount and used as a reference for the in vitro measurements. Another piece of substrate is placed on a plastic-covered foam block and product application is made to the “topography” side of the substrate (the rough side). The test composition (different commercially available dihydroxyacetone (DHA) containing lotions, 0.032 g) is applied evenly across a 4 cm×4 cm section of the substrate, which results in an application dose of 2 mg/sq. cm and rubbed into the substrate with a finger covered with finger cot. Afterwards, the sample is placed back on the balance where this time 0.032 g of the test polyamine compounds is added. After this treatment, the substrate is placed on a slide mount and placed into the 85% glycerin hydration chamber for 24 hours, 2 measurements are taken per sample and averaged. After that period, the sample (still on the slide) is measured for any color development. When the measurements are done, the slides are placed back into the hydration chamber for another 24 hour cycle to then taken out to measure the color development after 48 hours. Color development in controls (DHA-containing lotions only) is compared with color development in samples that are treated with the instant polyamine derivatives.

The samples are evaluated for total color change, Delta E, from measurements of the treated area subtracted from measurements of the untreated area. The color values are measured on a Datacolor Spectraflash SF650X spectrophotometer using D65 illuminant with 10° observer.

Delta E is calculated according to the following formula:

Delta E=[(L_{f}−Lᵢ)²+(a_{f}−aᵢ)²+(b_{f}−bᵢ)²]^{1/2}
f=final reading after specified time interval
i=initial reading at time=0 hours

### EXAMPLE 53

### Hydrophilic Modification of Skin-Like Surface

Contact angles are measured on Kruss DSA-10 Contact Angle Measuring System according to the static or sessile drop method and using water as a probe solution (as described in Kruss DSA1 v1.80 Drop Shape Analysis User Manual V020902—Kruss GmBH, Hamburg, 2002). VITRO-SKIN substrate is prepared according to the procedure described above. A piece of hydrated substrate is mounted on a glassless slide and air-dried for 15 minutes. It is used as a reference (or blank) for untreated VITRO-SKIN during the contact angle measurements. The Instant Compounds, as a 1 wt % solution dissolved in deionized water, are applied on the “skin topography” side of the VITRO-SKIN placed on plastic-covered foam block. Exactly 0.032 g of test product is applied evenly across a 4×4 cm section of the VITRO-SKIN that resulted in a standardized product application dose of 2 mg/sq.cm. Immediately after product application, the product is rubbed into the film with a finger covered with fingercot. After that, the film is placed on a slide mounted and air-dried for 15 minutes. Before measurements, VITRO-SKIN is removed from the slide mount and cut to several small pieces, which are used for the measurements. The use of small size pieces is necessary to assure its flat position of the film on the sample table. Extra care is taken to assure that the rough side was up and the film was flat. Contact angle measurements are conducted expeditiously—within approximately 1 minute.

| Instant Polyamine Compound | Contact Angle |
|---|---|
| Blank Vitro Skin | 101.7 |
| Unsubstituted Polylysine | 57.5 |
| Instant Example 1 | 42.4 |
| Instant Example 2 | 32.4 |
| Instant Example 3 | 26.1 |

The depressed contact angles found for the IN VITRO skin samples treated with the instant polyamine compound aqueous solutions indicate that the surface is hydrophilically modified even better that unsubstituted polylysine itself. The Instant Compounds produce a hydrophilic modification of the VITRO-SKIN surface that indicate their potential moisturization and good sensory properties.

### EXAMPLE 54

### Evaluation of Sunless Tanning Formulation

Following the test protocol described in Example 52, the instant compounds are evaluated for accelerated color development using a commercial sunless tanning formulation. The commercial formulation used is AVEENO Active Naturals Skin Tones. The instant compounds are tested as a 1 wt % solution in butylene glycol.

| | Delta E | Delta E |
|---|---|---|
| Instant Polyamine Compound | 24 hours | 48 hours |
| Blank Vitro Skin | 0 | 0 |
| Commercial Formulation Alone | 1.89 | 3.93 |
| Commercial Formulation/ | 3.83 | 8.12 |
| Instant Example 3 | | |
| Commercial Formulation/ | 4.13 | 8.30 |
| Instant Example 1 | | |

The data demonstrates the efficacy of the sunless tanning composition when used in conjunction with the instant substituted polyamine compounds. This is demonstrated by the increase in color generation, Delta E.

### EXAMPLE 55

### Comparative Evaluation in a Sunless Tanning Formulation

Following the test protocol described in Example 52, the instant compounds are evaluated for accelerated color development using a commercial sunless tanning formulation. The commercial formulation used is JERGENS Natural Glow (Medium/Tan Skin Tones. The instant compounds are tested along with their unsubstituted counterparts.

| | Delta E after |
|---|---|
| Instant Polyamine Compound | 24 hours |
| Commercial Formulation/ | 1.48 |
| 2 wt % polylysine in deionized water | |
| Commercial Formulation/ | 1.56 |
| Propylene Glycol | |
| Commercial Formulation/ | 1.60 |
| 2 wt % Compound A in propylene glycol | |
| Commercial Formulation/ | 3.4 |
| 2 wt % Instant Example 1 in deionized water | |

Polylysine is purchased from Chisso.

Compound A is 2-[2′-Hydroxy-3′-t-butyl-5′-(2-methoxycarbonyl ethyl)phenyl]-benzotriazole.

The data demonstrates the efficacy of the sunless tanning composition when used in conjunction with the instant substituted polyamine compounds. This is demonstrated by the increase in color generation, Delta E, even when compared with the unsubstituted counterparts.

### EXAMPLE 56

### Evaluation of Sunless Tanning Formulation

Following the test protocol described in Example 52, the instant compounds are evaluated for accelerated color development using a commercial sunless tanning formulation. The commercial formulation used is OLAY Body Quench Radiance Reviver. The instant compounds are tested as a 1 wt % solution in butylene glycol.

| | Delta E |
|---|---|
| Instant Polyamine Compound | 24 hours |
| Blank Vitro Skin | 0 |
| Commercial Formulation Alone | 3.35 |
| Commercial Formulation/ | 5.31 |
| Polylysine | |
| Commercial Formulation/ | 5.64 |
| Instant Example 3 | |

Polylysine is purchased from Chisso.

The data demonstrates the efficacy of the sunless tanning composition when used in conjunction with the instant substituted polyamine compounds. This is demonstrated by the increase in color generation, Delta E.

### EXAMPLE 57

### Evaluation of Sunless Tanning Formulation

Following the test protocol described in Example 52, the instant compounds are evaluated for accelerated color development using a commercial sunless tanning formulation. The commercial formulation used is AVEENO Active Naturals Continuous Radiance Medium. The instant compounds are tested as a 1 wt % solution in butylene glycol.

| | Delta E | Delta E |
|---|---|---|
| Instant Polyamine Compound | 24 hours | 48 hours |
| Blank Vitro Skin | 0 | 0 |
| Commercial Formulation Alone | 2.27 | 4.56 |
| Commercial Formulation/ | 5.93 | 10.31 |
| Instant Example 2 | | |
| Commercial Formulation/ | 6.29 | 10.62 |
| Instant Example 1 | | |

The data demonstrates the efficacy of the sunless tanning composition when used in conjunction with the instant substituted polyamine compounds. This is demonstrated by the increase in color generation, Delta E.

### EXAMPLE 58

### Evaluation of Sunless Tanning Formulation

Following the test protocol described in Example 52, the instant compounds are evaluated for accelerated color development using a commercial sunless tanning formulation. The commercial formulation used is BANANA BOAT Summer Color Sunless Light/Medium. The instant compounds are tested as a 1 wt % solution in butylene glycol.

| | Delta E | Delta E |
|---|---|---|
| Instant Polyamine Compound | 24 hours | 48 hours |
| Blank Vitro Skin | 0 | 0 |
| Commercial Formulation Alone | 9.83 | 12.41 |
| Commercial Formulation/ | 12.52 | 15.81 |
| Instant Example 2 | | |
| Commercial Formulation/ | 12.61 | 16.74 |
| Instant Example 1 | | |
| Commercial Formulation/ | 13.66 | 17.75 |
| Instant Example 3 | | |

The data demonstrates the efficacy of the sunless tanning composition when used in conjunction with the instant substituted polyamine compounds. This is demonstrated by the increase in color generation, Delta E.

What is claimed is:

1. A cosmetic and/or dermatological composition comprising
i) a sunless tanning agent,
ii) a substituted polyamine compound of formula (I) 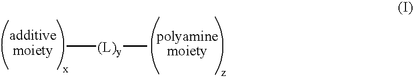
wherein
x and y are each independently greater than or equal to 1;
z is 1 to 5;
L is independently of each other a direct bond or a chemical linking group;
additive moiety is independently selected from the group consisting of antioxidant, ultraviolet light absorber, hindered amine light stabilizer, hydroxylamine stabilizer, nitrone stabilizer, amine oxide stabilizer, and benzofuranone stabilizer or mixtures thereof;
polyamine moiety is independently selected from the group consisting of polyethyleneimine, polyaminoacids, polyvinylamine, and oligomeric ethylene amines or mixtures thereof; and
iii) a cosmetically acceptable adjuvant,
with the proviso that in formula (I) of component ii) the additive moiety is covalently attached to said polyamine moiety through said chemical linking group.

2. A composition according to claim 1, wherein the self-tanning agent of component i) is a mono- or polycarbonyl compound.

3. A composition according to claim 2, wherein the self-tanning agent of component i) is selected form the group consisting of isatin, alloxan, ninhydrin, glyceraldehyde, mesotartaric aldehyde, glutaraldehyde, erythrulose, a pyrazoline-4,5-dione derivative, dihydroxyacetone (DHA) and a 4,4-dihydroxypyrazoline-5-dione derivative or mixtures thereof.

4. A composition according to claim 3, wherein the self-tanning agent of component i) is dihydroxyacetone.

5. A composition according to claim 1, wherein the concentration of the self-tanning agent of component i) is from 0.01% to 50% by weight relative to the total weight of the composition.

6. A composition according to claim 5, wherein the concentration of the self-tanning agent of component i) is from 0.1% to 20% by weight relative to the total weight of the composition.

7. A composition according to claim 6, wherein the concentration of the self-tanning agent of component i) is from 0.5% to 10% by weight relative to the total weight of the composition.

8. A composition according to claim 1, wherein for the substituted polyamine compound of formula (I) of component ii)
L is selected from the group consisting of a direct bond, —OCO—, —COO—, —O—, —CONH—, —CONR—, —NHCO—, —NRCO—, —CO—, —NH—, —NR—, —S—, —SO—, SO₂—, —CSO—, —COS—, —CSS—, and divalent hydrocarbylene group;
where R is a hydrocarbyl group.

9. A composition according to claim 1, wherein the composition comprises an additional colouring agent which is an insoluble extract of a red wood of genus Pterocarpus or of genus Baphia.

10. A composition according to claim 1, wherein the composition comprises an additional colouring agent which is an iron oxide nanopigment, the mean size of the nanopigment being less than 100 nm.

11. A composition according to claim 1, wherein the composition comprises at least one cosmetically acceptable adjuvant which is a fatty substance, an organic solvent, an emulsifier, an ionic or nonionic thickener, a softener, an antioxidant, a free-radical scavenger, an opacifier, a stabilizer, an emollient, a silicone, an .alpha.-hydroxy acid, an antifoam, a moisturizer, a vitamin, an insect repellent, a substance P antagonist, an anti-inflammatory agent, a fragrance, a preserving agent, a surfactant, a filler, a polymer, a propellant or an acidifying or basifying agent.

12. A composition according to claim 1, wherein the composition additionally comprises at least one organic photoprotective agent and/or at least one inorganic photoprotective agent that is active in the UVA and/or UVB range.

13. A composition according to claim 12, wherein the organic photoprotective agent is a 1,3,5-triazine derivative, a dibenzoylmethane derivative, a cinnamic derivative, an anthranilate, a salicylic derivative, a camphor derivative, a benzophenone derivative, a .beta.,.beta.-diphenylacrylate derivative, a benzotriazole derivative, a benzalmalonate derivative, a benzimidazole derivative, an imidazoline, a bis-benzazolyl derivative, a p-aminobenzoic acid (PABA) derivative, a methylene bis(hydroxyphenyl)benzotriazole derivative, a screening polymer, a screening silicone, a dimer derived from alpha.-alkylstyrene or a 4,4-diarylbutadiene, or mixtures thereof.

14. A composition according to claim 13, wherein the organic photoprotective agent is: ethylhexyl salicylate, butyl methoxydibenzoylmethane, ethylhexyl methoxycinnamate, octocrylene, phenylbenzimidazolesulphonic acid, terephthalylidenedicamphorsulphonic acid, benzophenone-3, benzophenone-4, benzophenone-5,4-methylbenzylidenecamphor, benzimidazilate, anisotriazine, ethylhexyltriazone, diethylhexylbutamidotriazone, methylenebis(benzotriazolyl)tetramethylbutylphenol, drometrizole trisiloxane, 2,4,6-tris(diisobutyl 4′-aminobenzalmalonate)-s-triazine, or mixtures thereof.

15. A composition according to claim 12, wherein the inorganic photoprotective agent is a coated or uncoated metal oxide pigment or nanopigment.

16. A composition according to claim 15, wherein the organic photoprotective agent is a coated or uncoated nanopigment of titanium oxide, iron oxide, zinc oxide, zirconium oxide, cerium oxide, or mixtures thereof.

17. A composition according to claim 12, wherein the organic photoprotective agent and/or inorganic photoprotective agent is present in the composition in proportions ranging from 0.1% to 20% by weight relative to the total weight of the composition.

18. A composition according to claim 17, wherein the organic photoprotective agent and/or inorganic photoprotective agent is present in the composition in proportion ranging from 0.2% to 15% by weight relative to the total weight of the composition.

19. A composition according to claim 1, wherein the concentration of the substituted polyamine compound of formula (I) of component ii) is from 0.01% to 50% by weight relative to the total weight of the composition.

20. A composition according to claim 19, wherein the concentration of the substituted polyamine compound of formula (I) of component ii) is from 0.1% to 20% by weight relative to the total weight of the composition.

21. A composition according to claim 20, wherein the concentration of the substituted polyamine compound of formula (I) of component ii) is from 0.5% to 10% by weight relative to the total weight of the composition.

22. A composition according to claim 1, wherein the composition is in the form of a nonionic vesicular dispersion, an emulsion, a cream or a triple emulsion, a milk, a gel, a cream-gel, a suspension, a dispersion, a mousse or a spray.

23. A composition according to claim 22, wherein the emulsion is an emulsion of water-in-oil type or of oil-in-water type, and the triple emulsion is a W/O/W or O/W/O emulsion.

24. A method for tanning or browning skin, which comprises applying to the skin an effective amount of the composition according to claim 1.
